(19) United States
(12) Patent Application Publication
(10) Pub. No.: US 2015/0112613 A1
(43) Pub. Date: Apr. 23, 2015

(54) System for Measurement and Analysis of Movement of Anatomical Joints and/or Mechanical Systems

(71) Applicant: Malcolm J. Smith, Deer River, MN (US)

(72) Inventor: Malcolm J. Smith, Deer River, MN (US)

(21) Appl. No.: 14/585,144

(22) Filed: Dec. 29, 2014

Related U.S. Application Data
(62) Division of application No. 13/097,997, filed on Apr. 29, 2011.
Application No. 13/097,997 is a continuation-in-part of application No. 12/589,796, filed on Oct. 27, 2009, now abandoned.
(60) Provisional application No. 61/108,838, filed on Oct. 27, 2008.

(51) Int. Cl. G01L 3/02; A61B 5/00; A61B 5/11

(52) U.S. Cl. CPC G01L 3/02; A61B 5/1118; A61B 5/1123; A61B 5/7445; A61B 5/4528; A61B 5/6831
USPC 702/44

(57) ABSTRACT

A system for measuring and analyzing movement or force in conjunction with sports, physical fitness or therapy. Real-time displays of range of movement (or force) information are calibrated for optimal visibility and/or resolution based on the actual range of a given exercise. Also disclosed is a balance indicator showing in real-time whether one side of the body is being favored over the other. Also disclosed are timing windows that indicate the desired timing of an exercise in real-time. Also disclosed are ‘breadcrumbs’ visually depicting (during and after completion of an exercise) a user's history of speed and range.

### RELATED APPLICATIONS

This application is a divisional of co-pending U.S. patent application Ser. No. 13/097,997 filed on Apr. 29, 2011 with the same title, which application was in turn a continuation-in-part of U.S. patent application Ser. No. 12/589,796 filed on Oct. 27, 2009 with the same title, and claims the benefit of U.S. provisional patent application Ser. No. 61/108,838 filed Oct. 27, 2008 and entitled “A Wired or Wireless Real-time System Incorporating the use of Software, Firmware and Hardware to Measure the Degree of Movement of a Human or Animal Anatomical Joint, or the Degree of Movement of Any Mechanical Device as used in Physical fitness, Sports or Physical Therapy.” The disclosures of the foregoing patent applications are incorporated by reference as if fully set forth herein.

### FIELD OF THE INVENTION

The present invention relates to the fields of sports, sports medicine, physical fitness or physical therapy, and more particularly to a system for measuring and analyzing extension and/or flexion of anatomical joints, and/or rotary and/or linear movement of a mechanical system or machine.

### SUMMARY OF THE INVENTION

The present invention comprises a system for measuring and analyzing movement (extension and/or flexion) of a human or animal anatomical joint, and/or the linear and/or rotational movement of a mechanical system, in conjunction with sports, physical fitness, or physical therapy. Sensors can be attached externally to an anatomical joint, and/or to the moving parts of one or more mechanical systems. Information from such sensors is digitized, and software (e.g., on a personal computer, PDA, embedded computer, cellphone, etc.) is used to display, archive, compare, and analyze the sensor information.

The joint or machine movement information can be analyzed and responded to in real-time and/or archived for later comparison and analysis. Movement cycles for each exercise performed preferably can be pre-defined and stored in a file system on removable or non-removable storage devices, so that trends and performance statistics can be reviewed. For example, an exercise performed today can be compared with one done a week ago, a month ago, or even years ago.

Real-time range of movement (or force) information preferably may be displayed via gauges, along with other information such as total weight moved, sets to do and completed, repetitions to do and completed, elapsed time for each component of the movement cycle. The real-time display can help a user correct an exercise in real-time. The gauges or readouts also preferably may be calibrated to optimize their visibility and/or resolution based on the actual range of motion of a given exercise.

In a separate and independent aspect of the invention, the system can indicate to the user in real time if they are favoring one side of the body over the other, so the user can compensate with the weaker side of the body and reduce the tendency to exert more power and/or range of motion on the user's dominant side.

In another separate and independent aspect of the invention, timing windows can visually depict the desired timing of an exercise a user is performing, helping the user to perform the exercise with the desired timing.

In yet another separate and independent aspect of the invention, ‘breadcrumbs’ can visually depict the user's history of speed and range of motion during and after completion of an exercise, to indicate if the user is performing repetitions too fast or too slow.

### BRIEF DESCRIPTION OF THE DRAWINGS

FIG. 1 shows sensor supports attached to a human elbow and knee extremity.

FIG. 2 shows an extremity elbow/knee textile support.

FIG. 3 shows a vacuum-sealed bend sensor.

FIG. 4 shows a transmitter and receiver system overview.

FIG. 5 shows battery power management logic.

FIG. 6 shows a sensor calibration screen.

FIG. 7 shows a main sensor information display screen.

FIG. 8 shows detail trending graphs.

FIG. 9 shows summary trending graphs.

FIG. 10 shows a database setup and configuration screen.

FIG. 11 shows the main exercise screen as displayed by a touchscreen LCD display.

FIG. 12 shows another exercise screen.

FIG. 13 shows another exercise screen.

FIGS. 14-16 are close-ups of a balance indicator display.

FIGS. 17-19 are close-ups of a left range indicator display, showing a timing window.

FIGS. 20-22 are close-ups of a left range indicator display, showing ‘breadcrumbs.’

FIGS. 23-25 are close-ups of a left range indicator display, showing ‘count zones.’

FIG. 26 shows identification of the highest high and the lowest low of a range indicator display during calibration.

FIG. 27 shows the total un-calibrated range of a range indicator display during calibration.

FIG. 28 shows the range-of-motion of a calibrated range indicator display.

### DETAILED DESCRIPTION OF EMBODIMENTS

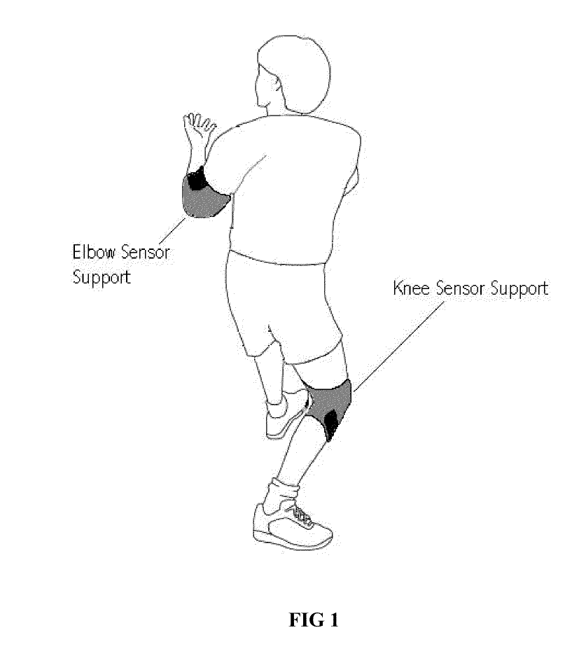

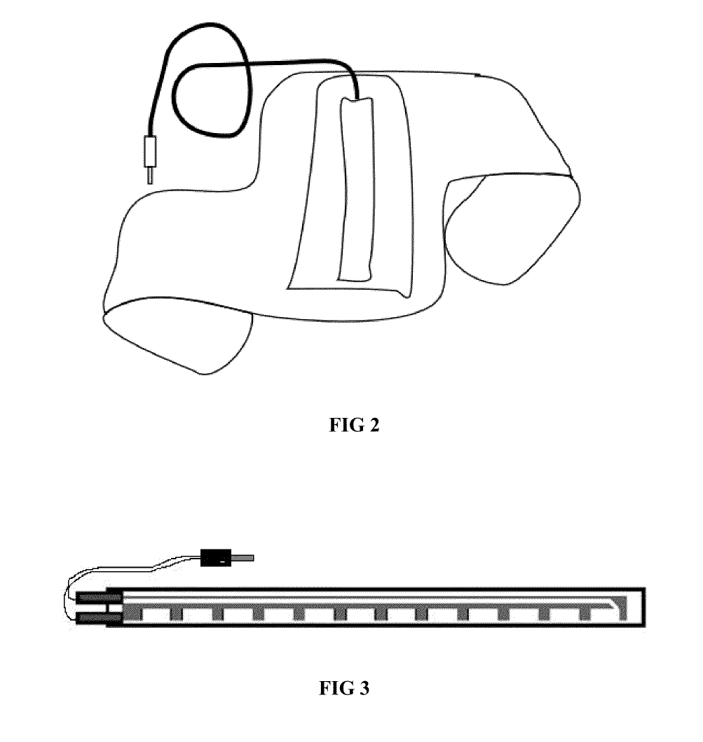

An embodiment of a system according to the invention in the context of an application relating to physical therapy, health, and/or fitness may include one or more sensors incorporated into aerated neoprene and nylon supports that are adjustable, lightweight and comfortable, and worn on the body extremities as shown in FIG. 1. As shown in FIG. 2, the sensors are secured and aligned to each neoprene and nylon support with an elastic layer covering and holding the sensor firmly against the support, and preventing the sensor from moving out of alignment while not restricting or encumbering the range of motion. As shown in FIG. 3, the sensors are preferably vacuum-sealed to eliminate dirt and moisture from entering the sensor and potentially damaging or affecting sensor signal integrity. Such a moisture-resistant system would accommodate the analysis of joints while exposed to, or submersed in water. The sensors can utilize resistive characteristics that vary in response to an external physical force (such as a variable resister or strain gauge), or could be based on any other technology for measuring rotary or linear movement, such as optical incremental encoders, magnetic incremental encoders, potentiometers, or bend-sensors. The sensors may be secured externally to the body using textiles, hook and loop and plastics, and are attached to the mechanical systems as appropriate (e.g., with a mechanical structure that allows the sensor to make contact with one or more moving parts of the target equipment). The sensor supports may be primarily constructed of nylon or neoprene, textiles, ferrous and non-ferrous metals, plastics, and/or any other materials suitable for securing the sensor to a joint or machine without impeding mobility or performance, with the size and type of materials used depending on the requirements of the joint to be analyzed. Possible points of contact for a sensor may include a moving part of an exercise machine such as a pulley, gear, piston, cable.

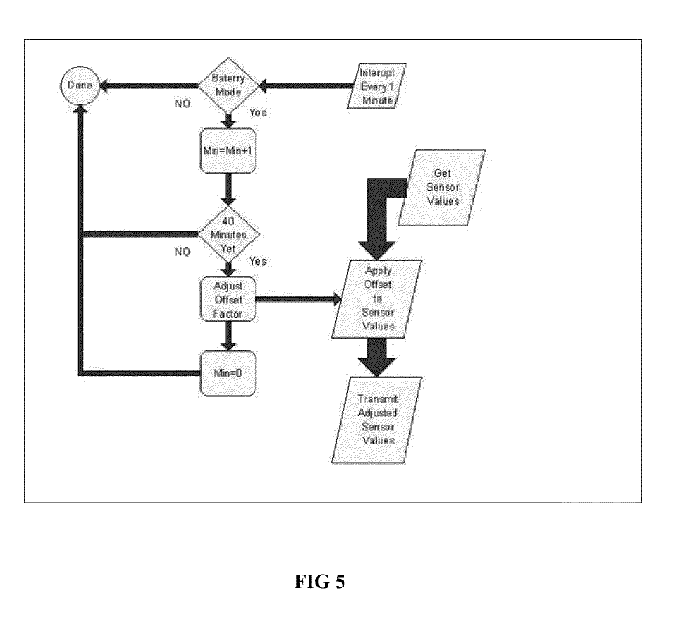

A radio frequency (RF) transmitter can be employed to convey sensor information, enabling the user to move freely. In some applications, the sensors could be wired to the processing electronics (e.g., so the entire system can take power from a standard AC outlet). The sensors can be connected to the signal processing electronics using fine-gauge flexible electrical wire with small quick-disconnect jacks, enabling processing electronics to be removed without removing the sensors. Sensor connection wires can be placed under or clipped to clothing to avoid unintentional disconnection. The transmitter is preferably located on or near the user, with an external membrane keypad for user interaction. If the transmitter is battery-powered, then software (e.g., residing in flash memory within the transmitter module's microprocessor) routines monitor the battery condition as shown in FIG. 5, re-calibrating sensor input as necessary to maintain consistent sensor output levels. Particularly in a wireless configuration, a slave microcontroller (MCU), microprocessor unit (MPU), or CPU may be provided to communicate with sensors; in any case, the host (e.g., an external personal computer, server, PDA, or an integrated standalone device including a display) preferably comprises a MCU, MPU, or CPU, and the system further includes memory, a real-time clock, and firmware run by the host to provide communications between the sensors and the host, generate real-time information, display user options, and digitally store data, with a user interface such as touch screen, voice recognition, mechanical or electrical switches, etc.

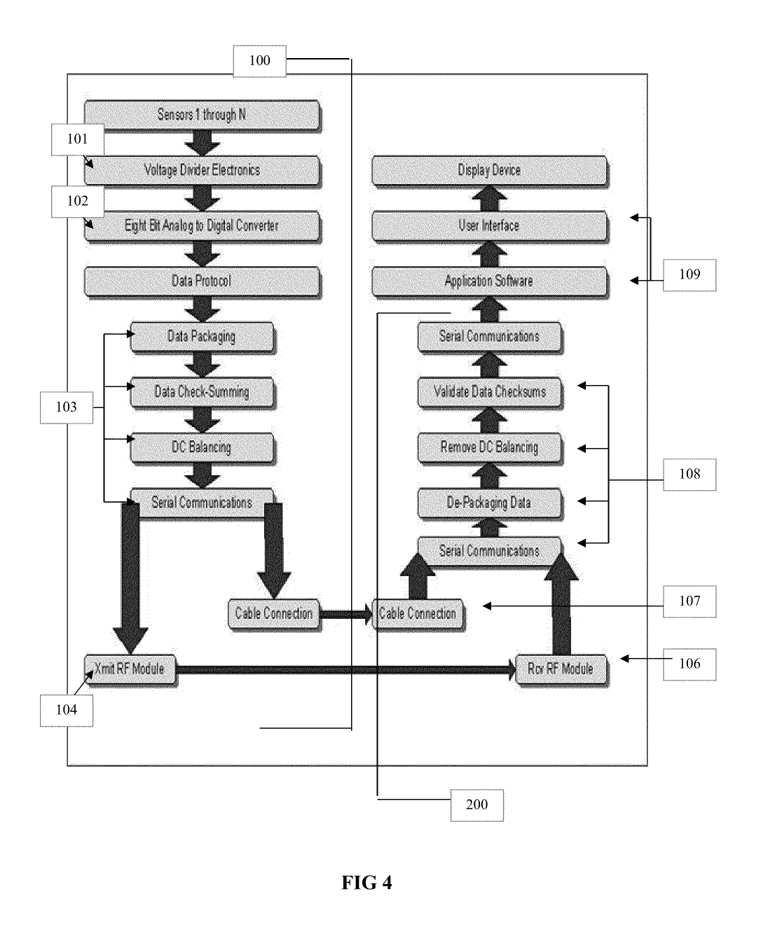

FIG. 4 shows the sensor signal processing flow for the transmitter 100 and the receiver 200. The system is a multiple stage system, the first stage being a voltage divider circuit 101 that attaches directly to the sensors and converts the input from the sensor into an analog signal (e.g., in the range of 0-5 volts DC) in direct proportion to the amount of sensor flexion.

The second stage is the conversion of the analog signal into digital 102 using an analog to digital (A/D) converter incorporated in a microprocessor unit, e.g., with eight, ten, twelve or more bits of resolution for a digital range of zero through 255, 1024, 4096 etc.

The third stage is data protocol 103, which includes data packaging, data check-summing, DC balancing, and serial communications. Data packaging is the process of prefixing and suffixing the signal data into a data packet, which comprises a specified number of eight-bit bytes conveying three main components: (1) a packet header, including a unique identification number and other system information; (2) sensor information for each of the sensors; and (3) a packet trailer, including the data packet checksum. Data can be checked by summing the original data bit with the interleaved DC balancing bit (as described further below); if that sum is not binary one, the data is deemed corrupt and discarded. DC balancing can be employed to facilitate RF data transmission and reception by ensuring that no more than two binary bits of the same type are transmitted in sequence; an additional eight-bit byte is added to every data byte in the complete packet, and integrated by interleaving a complementary bit next to each bit of the original eight-bit byte. The finished data packet with DC balancing and check-summing is then serially communicated.

The fourth stage is serial transmission of the data packets to a receiver via an FCC-compliant RF transmitter module 104 or a cable 107. All of the proceeding processes are performed in software that is executed on power-up of the transmitter module.

The fifth stage is serial reception of the transmitted data packets via an FCC-compliant RF receiver module 106 or cable 107.

Stage six includes de-packaging data 108, removing DC balancing, validating data check sums, and serial communications. After data is received from the transmitter, it is de-packaged. The header is first examined to verify its unique ID; if incorrect, the entire data packet is discarded. If the ID is correct for this receiving pair, header and trailer portions of the data packet are stripped, leaving only the DC-balanced sensor information. Next, the DC balancing information is removed by stripping every other binary bit from the data string. This data is then saved, and validated by summing each binary bit from each sensor reading and summing it with the associated previously-saved DC-balancing bit. (The data byte is deemed corrupt and discarded if the sum is not binary one). This process is repeated for each sensor reading until the entire data portion of the packet has been processed. Finally, the valid sensor readings are sent (e.g., using RS232 protocol) to the attached display unit.

The seventh stage relates to application software 109, which processes the received sensor information. The software is preferably adapted to facilitate the particular type of use without need for modifying hardware configurations. The application software 109 (which could be run locally or accessed remotely, e.g., via connection over the internet) allows one or more users to set up a workout schedule, including the day of the week, the approximate time (AM or PM) that a workout will be performed, and the type of exercise, number of sets, and reps and weights for each set, for each exercise to be performed on the respective days and times. This information is saved (e.g., onto a USB flash drive, which the user can connect to the system at the beginning of a workout), and the system can display workout schedules based on the current date and time, ranking them from best to worst match. For example, if it is Wednesday and a user has defined workout schedules for Monday, Wednesday, and Friday, the system will place the Wednesday schedule at the top of the selection screen.

When the user has selected a schedule, the system prompts the user to set up for the first exercise in the group. Setup information includes the name of the exercise, where the user sits or stands with respect to the actual exercise machine, the total number of sets scheduled, the total number of repetitions for this set, and the weight to use for this set. When the user is ready, he/she presses a footswitch or touches the touch-screen display to begin the exercise, whereupon the system starts recording information about the exercise (e.g., on the user's USB flash drive). The system preferably records (e.g., with 0.01 second resolution) four data points for each applicable side of the body: 1) start of the repetition; 2) top of the exercise; 3) top and down; and 4) bottom of the exercise. A display (e.g., LCD touch screen) provides information to the user in real-time including the status of the current exercise (for left and/or right sides as applicable), the number of repetitions completed and remaining for the current set, and the number of sets completed and remaining.

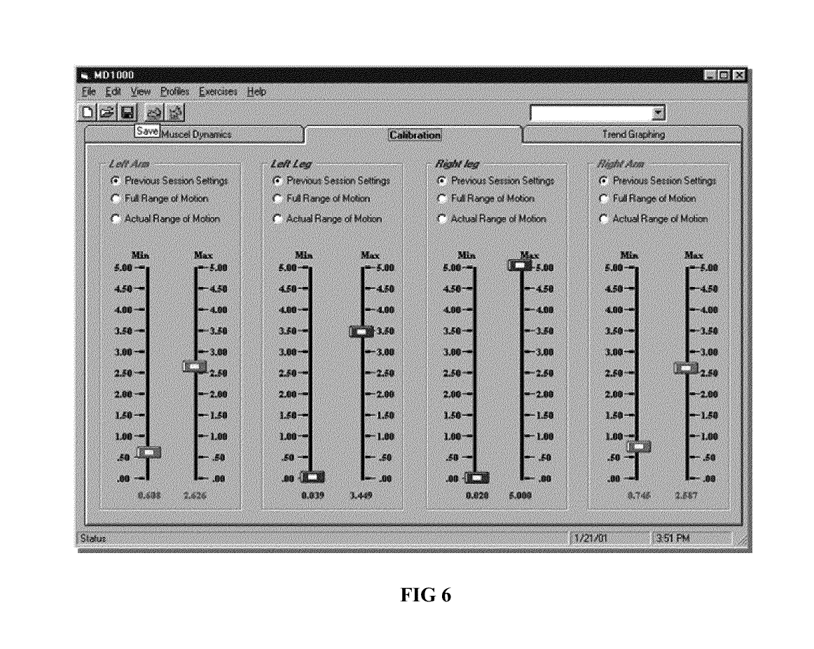

At the start of a monitoring session when the sensor supports are initially attached, the system is preferably calibrated to maximize the displayed range of motion (for isotonic exercises; range of force exerted measurements would be similarly read and calibrated in an alternative embodiment involving isometric exercises). The calibration process (which is described further below) may be manual or automatic; if automatic, the system preferably uses the first completed movement cycle to determine the begin and end range parameters. The software can set the calibrated minimum and maximum for each joint calibrated using a comprehensive calibration screen as shown in FIG. 6. After calibration, all data captured from the receiver module is buffered and the values adjusted based on the calibration factors. After completion of each exercise, the system automatically advances to the next scheduled exercise, although the user can manually select any exercise and change their order. The system records all completed exercises and will indicate to the user that the “Workout is complete” after every exercise in the current scheduled group has been completed. The system also can be configured to display user-defined messages (e.g., “Stretch for ten minutes” or “Fifteen minute cool-down”) during a workout.

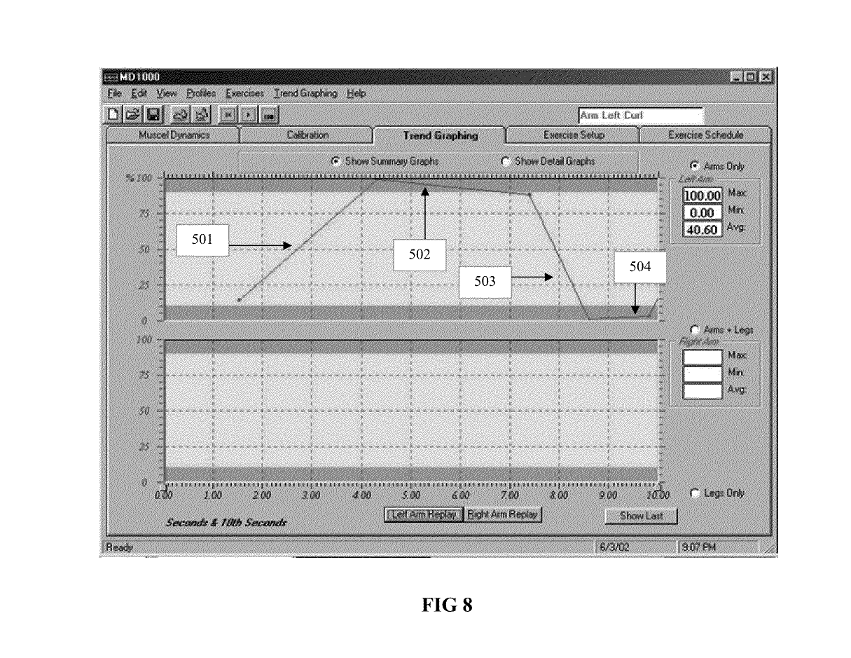

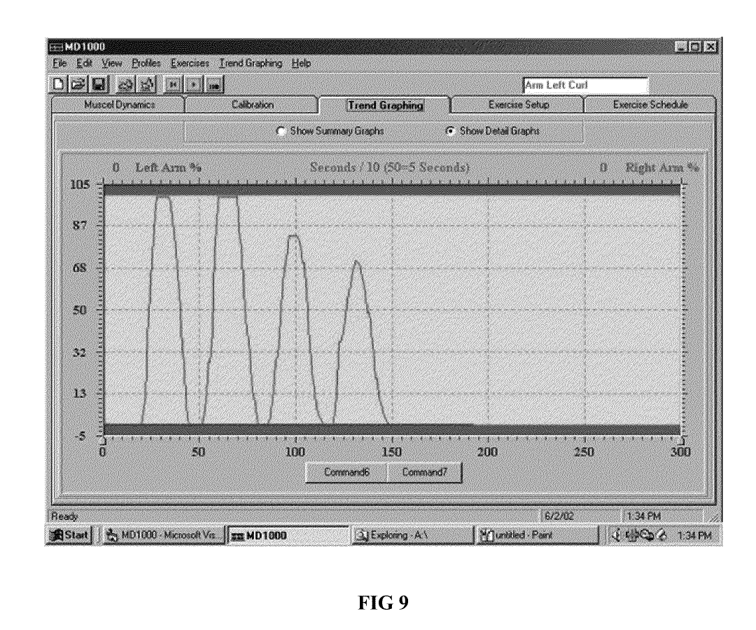

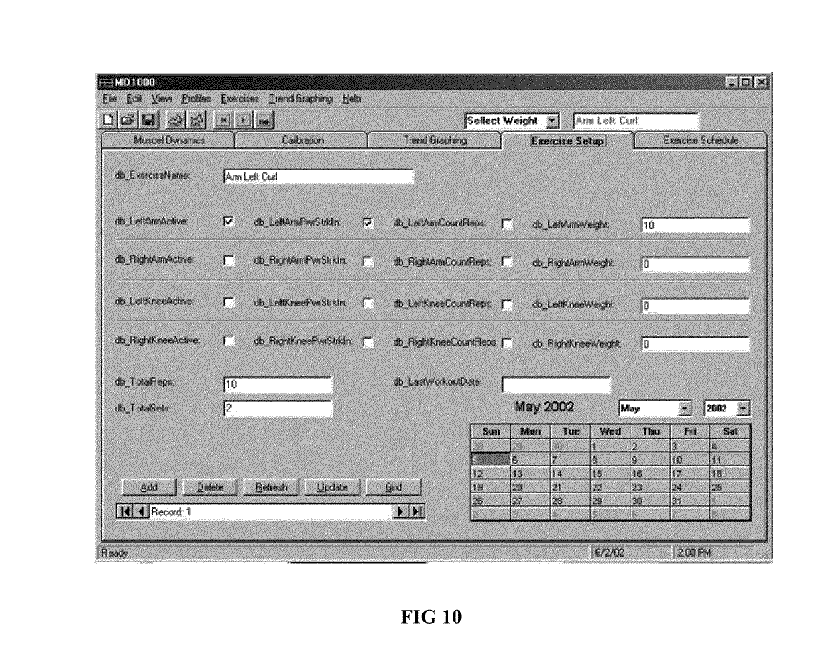

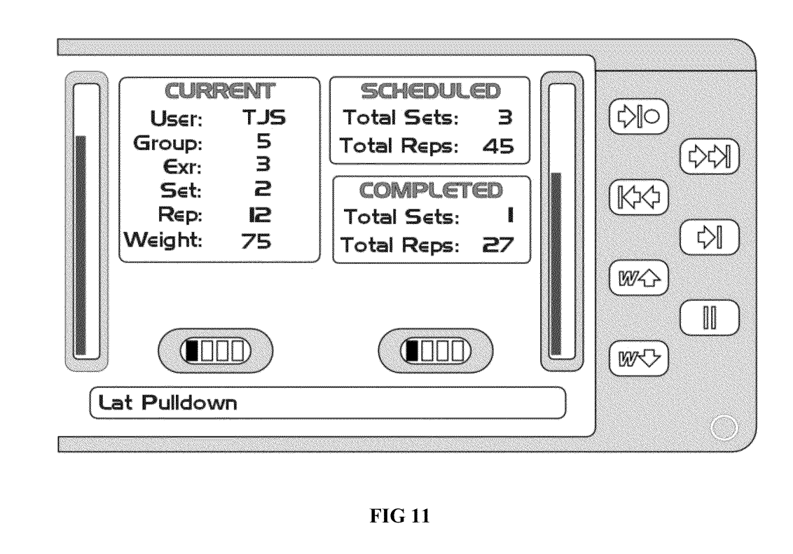

After the scheduled exercise group has been completed, the user can store the recorded information to a database (e.g., over the internet) and may perform analyses of it such as comparison with prior saved information. Joint movements can be compared with those completed hours, days, months, or years ago for complete trend analyses. A detail-graphing screen as shown in FIG. 8 can depict the dynamics of a movement cycle, which typically comprises four parts: 1) a limb moves away from its rest position to the eventual furthest extent of the movement 501; 2) a short pause with no movement 502; 3) the limb returns through the same path of travel to its original starting position 503; and 4) a period of no movement (rest) 504, which completes the movement cycle. As shown in FIG. 9, a trend summary displays performance for a movement set. As shown in FIG. 10, movement profiles can be configured in a database for future retrieval and use. For example, a complete exercise regimen can be set up with total weight and the number of repetitions to be performed. The recorded information also may be electronically conveyed, e.g., to a physical therapist or personal trainer, who could revise the exercise regimen appropriately and upload revised schedules to the system.

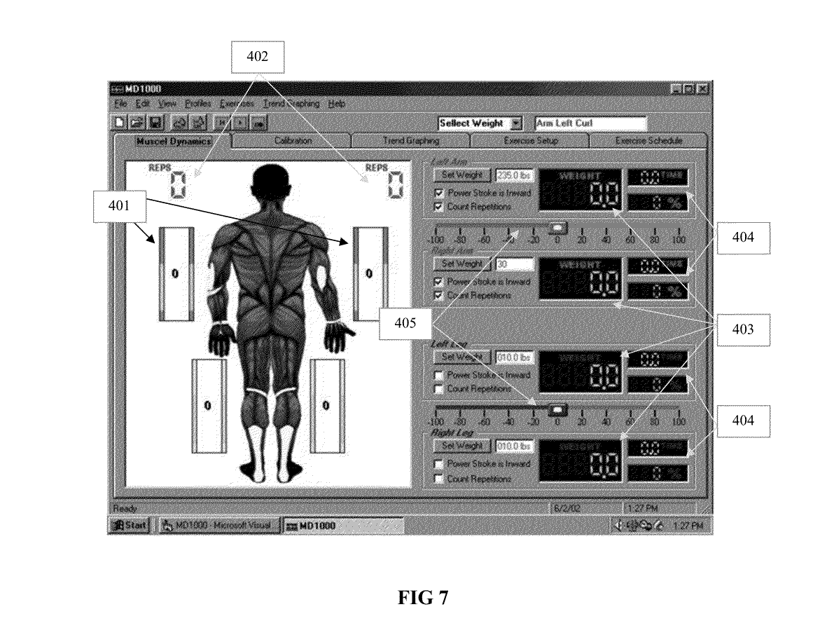

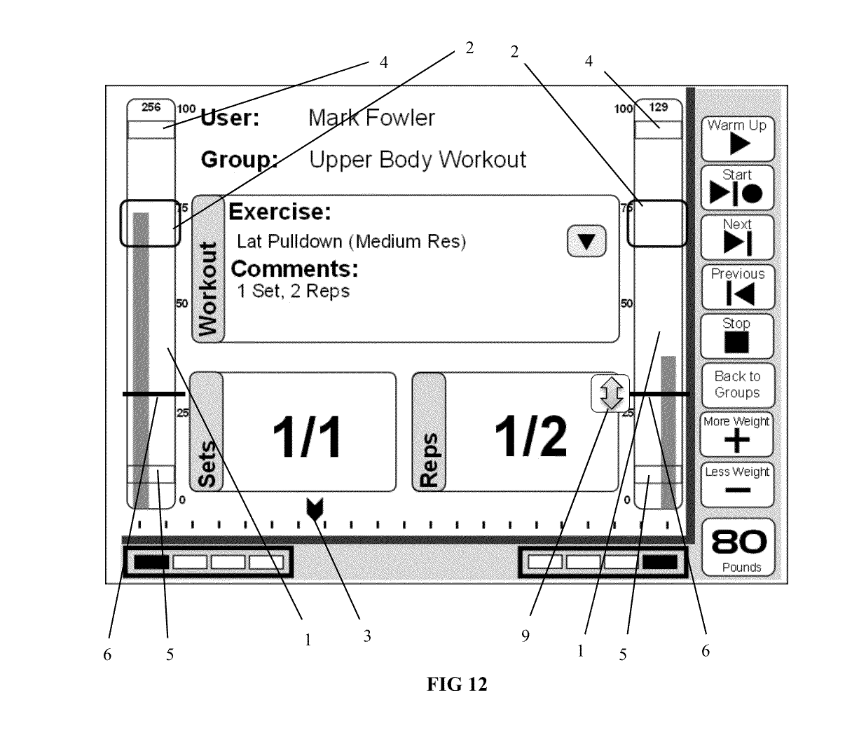

As shown in FIG. 12, range indicators 2 move up and down as the user performs the exercises. The top of the range indicator within the scale (0-100%) reflects the current progress of the user's range continuously for both the power and return stroke. As the depicted example is a parallel exercise (i.e., both sides of the body perform the same movements), both the left and right range indicators are displayed. A thermometer type scale 401 is used to display movement activity in real-time as shown in FIG. 7, with digital counters displaying total repetitions 402, total weight moved 403, and time elapsed for each completed repetition 404. A balance indicator 405 is displayed for parallel exercises, and moves from its center position toward the left or right to indicate if one side is ahead of the other. As shown in FIG. 12, a balance indicator 3 can be a horizontal gauge comprising a series of tick marks and a pointer that moves laterally above the tick marks, visually helping the user to use equal force and range on both the left and right side of the body. The pointer is on the left side in FIG. 12, showing the user favoring his left side (moving comparatively slower with his right arm); the range indicators 2 also show that the right arm was behind the left. The balance indicator is useful in compensating against tendency to use more power and range on the body's stronger side. The user observes the balance indicator while performing the exercise, and adjusts the force/range on either the left or right side of the body as needed to keep the pointer centered, reducing the likelihood of stress or injury due to improper exercise form.

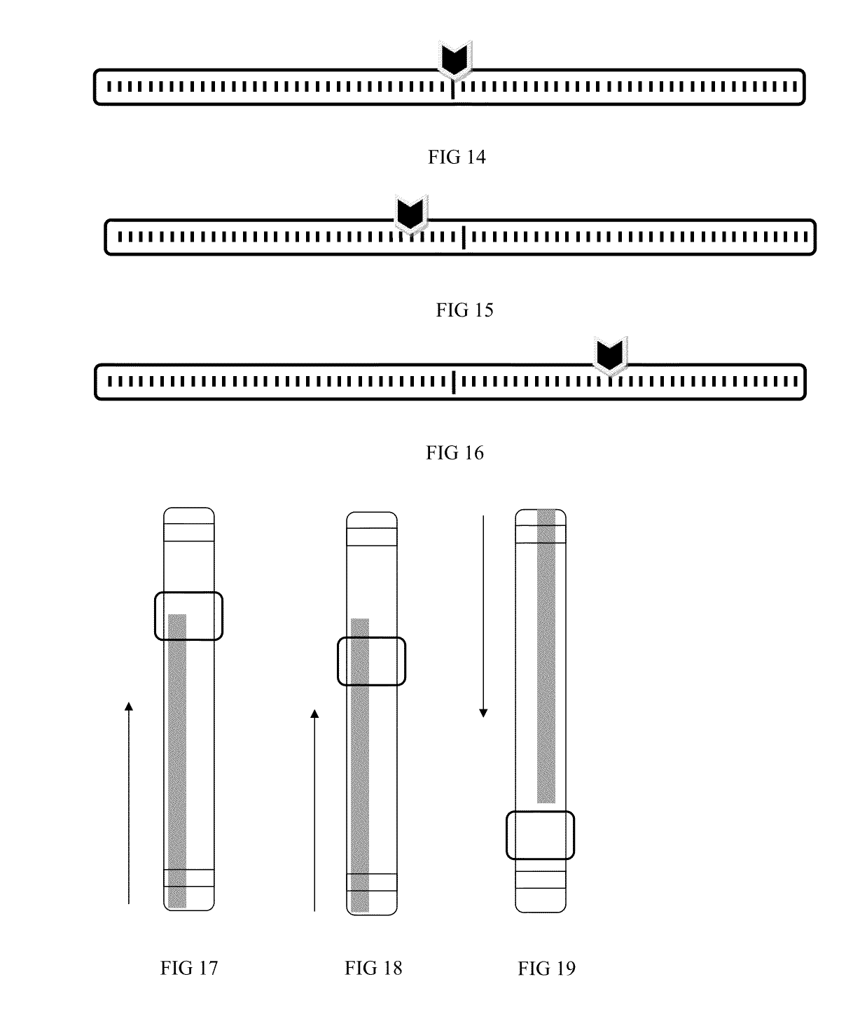

The balance indicator is directly associated with both the left and right range indicators. Before an exercise is started and the user is at rest, the range indicators will read zero, i.e., their leading edges will be at the very bottom of the scale. When the user begins the power stroke of the exercise, the range indicators' value will increase and their leading edges move upward proportionally to the arms' or legs' movement. The range of motion will be at its maximum when the user finishes the power stroke, and the leading edges of the range indicators correspondingly will be at their highest points for that repetition. As the user begins the return stroke, the leading edges of the range indicators will begin to move downward proportionally to the arms' or legs' movement until the user again reaches the rest position. The position of the pointer on the horizontal scale is the difference of the left range indicator and the right range indicator plus the offset, the offset being the numerical value of the scale's midpoint. For example, in FIGS. 14-16, the left-most position of the balance indicator scale is 0 and the right-most position is 1000, so the scale's midpoint—and offset—is 500. In FIG. 14, the left range indicator reads 450 and the right range indicator reads 400, so the balance indicator position=400−450+500=450, which is 50 units left of center, prompting the user to exert a bit more force on the right side of the body to compensate for the stronger left side. In FIG. 15, the left range indicator reads 800 and the right range indicator reads 650, so the balance indicator position=800−650+500=650, which is 150 points right of center, prompting the user to exert a good bit more force on the left side of the body to compensate for the stronger right side. In FIG. 16, the left range indicator reads 600 and the right range indicator reads 600, so the balance indicator position=600−600+500=500, which is the midpoint, indicating that equal force is being exerted on both sides of the body.

Rather than a horizontal linear gauge, the balance indicator alternately could take any number of other forms, such as a vertical pendulum type display or a horizontal light bar with square blocks of e.g., green, indicating that the user is in-range, with the colors changing to, e.g., orange and then red, as the indicator moves further to the outside of the display, indicating an increasing degree of imbalance via color. Audible alerts also could be used (e.g., “Please increase force on your right side”), with or without the visual display.

The timing windows shown in FIG. 12 are visual cues preferably comprising movable objects/shapes positioned over both the left and right range indicators. The timing windows are preferably semi-transparent so as not to obscure the range indicator's leading edge, and move up when the user begins the power stroke and then down when the user begins the return (i.e., the ‘negative’ or weight-lowering) stroke. The system is configured so that the timing windows move up and down at the optimal speed for each particular exercise, akin to a visual metronome depicting in real-time the optimum speed at which to perform a given exercise for both the power and return stroke of the exercise. The user attempts to keep the leading edges of the range indicators inside the timing windows during both the power and return stroke of each exercise, speeding up or slowing down as required to keep in sync with the timing windows.

The power and return strokes can be broken down into the following four phases: 1) transition from rest to the power stroke; 2) completion of the power stroke and transition to a rest period; 3) transition from rest to the return stroke; and 4) completion of the return stroke and transition to rest (after which the repetition is completed and the cycle may commence again). The durations of the rest periods can be set to specific values for each exercise type and/or user preference, preferably within limits that minimize the likelihood of injury. The timing windows are positioned at the bottom of the range indicators during the rest period or start of an exercise, and then begin moving upward at the start of the exercise. When the user completes the power stroke and transitions to rest, the timing windows pause at the top location of the range indicator. As the user transitions from rest to the return stroke, the timing windows begin moving downward. At the end of the return stroke, the timing windows stop at the bottom of the range indicator, ready for the next repetition to commence.

The speed can be set for each part of the exercise including the rest periods for both the start and end of the exercise, and may be programmed so that the timing windows start out slow and accelerate to the desired speed over a period of time, and provide a deceleration period when the user nears the ends of the power and return strokes so as to replicate a natural optimal dynamic for lifting and lowering a weight. The timing windows' speed and acceleration are also preferably programmable, with defaults pre-set based on the specific exercise and the user's motivation and experience levels. Since some exercises become more difficult simply by changing the speed at which they are performed, changing the speed of an exercise can be as important as changing the weight, range of motion, or number of repetitions.

FIGS. 17-19 show examples of the left range indicator and timing window during a bicep curl. In FIG. 17, the power stroke is being performed, and the timing window is centered over the leading edge of the range indicator, showing that the user is in sync with the timing window. In contrast, in FIG. 18, the timing window shows that the user is performing the power stroke too fast. In FIG. 19, the return stroke is being performed, and the timing window is ahead of the leading edge of the range indicator, indicating that the return stroke is being performed too slowly.

The system also preferably may track the number of times that the range indicator(s) led (too fast) or lagged (too slow) the timing window(s) during each repetition, and such information can be reviewed later to determine if the user's form and/or speed of the exercise should be changed. The timing windows also may be colored to enhance comprehension; for example, green could be used while the leading edge of the range indicator is well inside the timing window, yellow could be used as it approaches the boundary of the timing window, and red could be used when it is outside the timing window. The timing windows also alternately could be any other desirable shape.

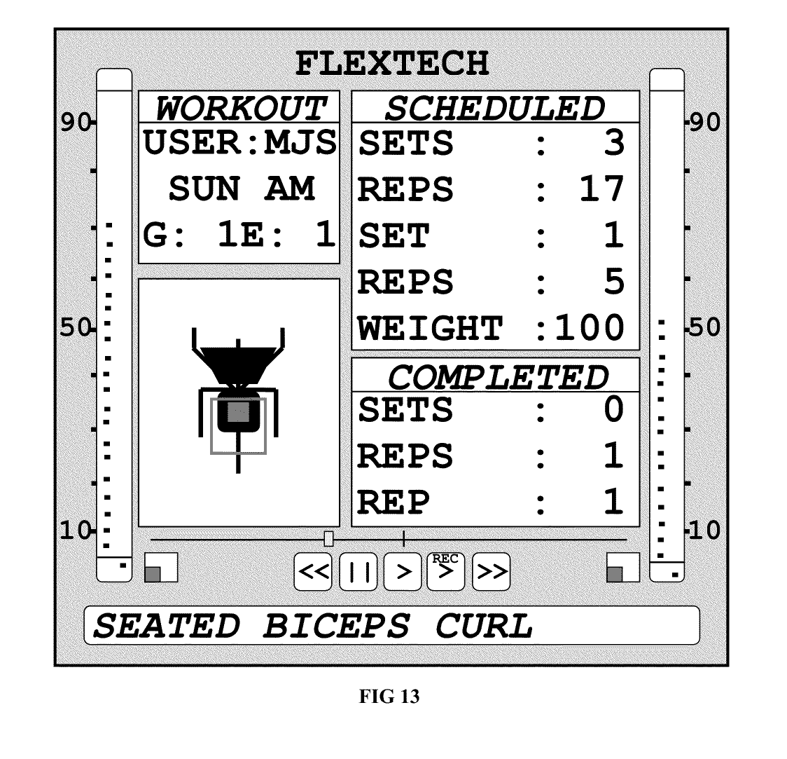

As shown in FIGS. 13 and 20-22, an embodiment of the system may utilize ‘breadcrumbs’ to indicate to the user if they are performing repetitions too fast or too slow. The breadcrumbs are a series of dots or dashes that show a user's history of speed and range of motion throughout an exercise. The breadcrumbs are placed at regular time intervals at a location on the scale to indicate the percentage of range accomplished thus far for the repetition. If the exercise is being performed too fast, the breadcrumbs will lie farther apart; if too slow, the breadcrumbs will lie closer together. This allows a user to review performance while a rep is being performed, and also after it has been completed. A short horizontal line or dot may be used to indicate where the leading edge of the range indicator is (but not on a constant basis; instead a predetermined pause, e.g., from 20 to 500 mS depending on the exercise, can preferably be used before any dash is drawn on the vertical display). When a user starts an exercise, the system loads a timer with a predetermined value (appropriate for the particular exercise, e.g., 50 or 100 mS) and counts that value down as the user moves through the power stroke. When the timer reaches zero, the system displays a dash on the gauge indicating the user's current extent of movement. The timer is reloaded with the predetermined value and the foregoing process repeats until the user completes the power stroke. Depending on the exercise, there may be as many as 50 to 500 dashes drawn over the duration of each of the power and return strokes.

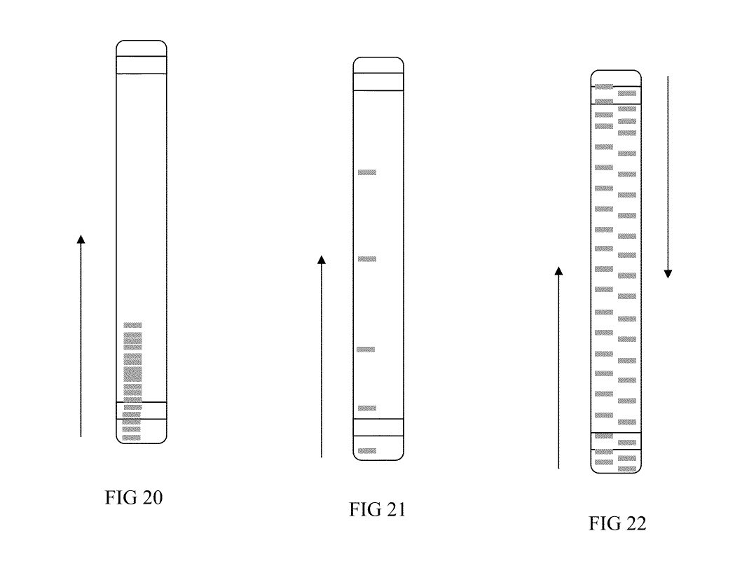

As an example, as shown in FIG. 20, if a left bicep curl is being performed very slowly, the dashes would be very close to each other. Conversely, as shown in FIG. 21, the dashes would be drawn farther apart if an exercise is being performed very quickly, since more distance or range is accomplished in the given time period. The user should attempt to have the breadcrumbs wind up regularly spaced and not too close together or far apart as shown in FIG. 22. After the exercise has been completed and before the next exercise is started, the user can review the breadcrumb patterns to see his/her performance.

Calibration is preferably provided to adjust all visual display scales to match a user's size and range. For example, on any given piece of exercise equipment, a tall person may have a different physical starting position than a short person. The system preferably may be configured to include a semi-automatic calibration method that automatically adjusts the maximum range of motion but requires the user to manually set the minimum range of motion, and/or a fully-automatic calibration method that automatically adjusts both the maximum and minimum range of motion. With either method, the calibration process is performed at least once for each exercise upon the first repetition of each set, after which the ensuing repetitions scheduled for the exercise set are performed in a calibrated state.

Using the semi-automatic or ‘fixed starting point’ calibration method, the user first gets into basic position (e.g., seated or standing) at the equipment where the exercise is to be performed. Second, the user selects the exercise to be performed (e.g., using touch screen or mouse and keyboard). Third, the user gets into a specific fixed starting position (typically a position in which a weight stack is minimally lifted from the at-rest position) for a repetition of the exercise being performed. Fourth, still in this starting (aka ‘at-rest’ or ‘zero’ or ‘home’) position, the user presses a start button (e.g., on a hand or foot switch), causing the system to save the physical location of the handles/weight stack and set it as the minimum range of motion value for each repetition of the current set. Fifth, the user commences the power stroke of the repetition, physically moving away from the previously-calibrated starting position. The system continually monitors the direction of the power stroke, visually showing this action as a rising bar in the range of motion indicator. As soon as the user reaches the end of the power stroke and begins reversing direction toward the starting position, the system logs the corresponding position as the highest high value and sets it as the maximum range of motion for this exercise and user. Sixth, the system uses these two data points to calculate a calibration factor to properly re-scale the un-calibrated range of motion data into calibrated data for display and storage. For example, assuming that the un-calibrated range of motion readout covers arbitrary units of 0 to 1024, and a user has (in the fourth step above) set a calibration starting value of 138 and the system identified (in the fifth step above) a maximum value of 766, the calibration factor would be the raw readout range divided by the actual range of motion, i.e., (1024−0)/(766−138)=1.63 (rounded). In that example, an un-calibrated range of motion reading of 314 would be displayed as 512 (which—being halfway in the calibrated full range of motion—would be displayed on the range-of-motion-indicators as a vertical bar starting at the bottom of the indicator and ending at the very middle of the indicator).

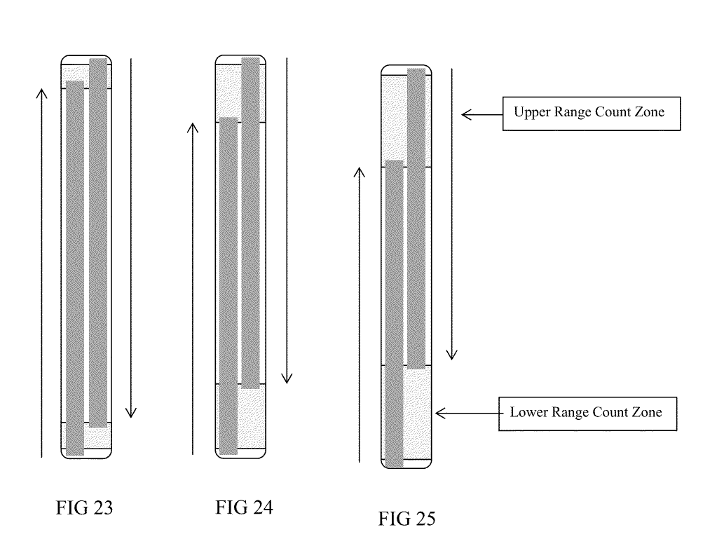

The system optionally also can be configured to provide upper count zones 4 and lower count zones 5 upon calibration, which may be displayed as colored semi-translucent rectangles respectively at the top and the bottom of the range-of-motion-indicators as shown in FIG. 12. The system counts a repetition as successful if it begins within the lower count zones, reaches or exceeds the upper count zones, and returns to within the lower count zones. For example, if the relative height of the count zones is set at 10% as in FIG. 23, each count zone would occupy 10% of the range-of-motion-indicator scale and allow the user to over- or under-extend by 10% on any repetition. The count zones' relative height can be user-specified (e.g., depending on the user's preferences, motivational level, specific workout goals etc.), with larger clips making it easier to have a repetition counted, and smaller clips requiring a longer and more exacting range of motion. The system may also be configured to permit count zone sizes to be modified globally for a user rather than individually for each exercise. It may be desirable for a user to start with very large count zones as a novice (e.g., 35% as shown in FIG. 25) and then slowly reduce them over time (e.g., to 20% as shown in FIG. 24 and then to 10% as shown in FIG. 23 or even lower) to make workouts increasingly difficult even without changing the weight or number of repetitions of each exercise.

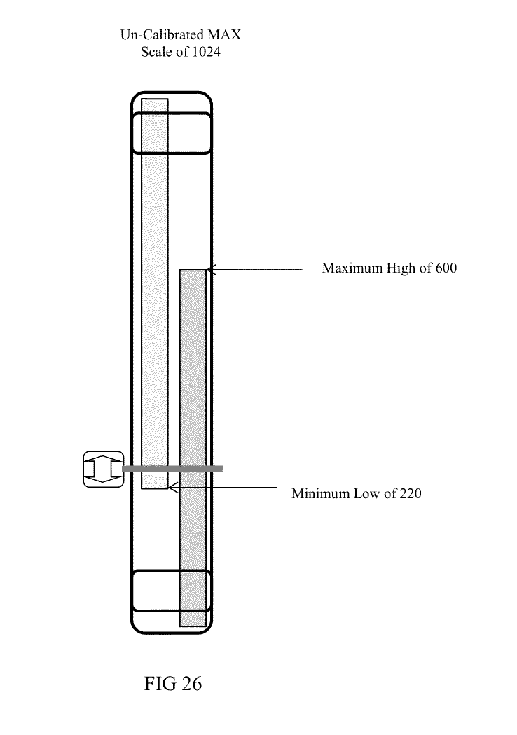

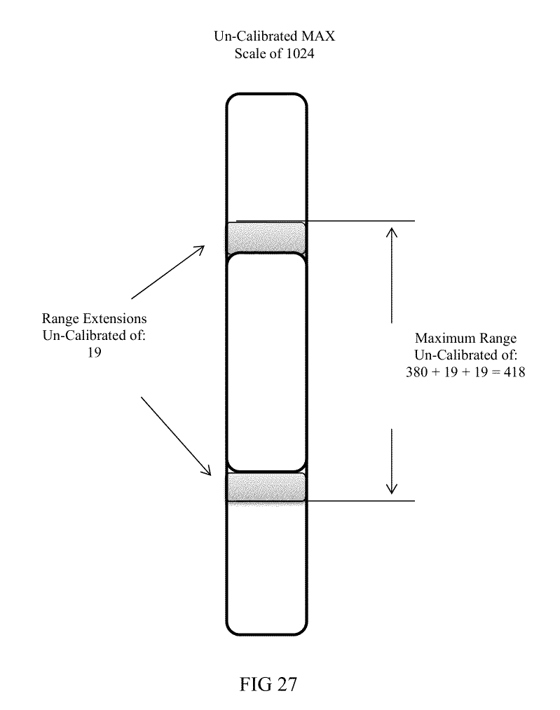

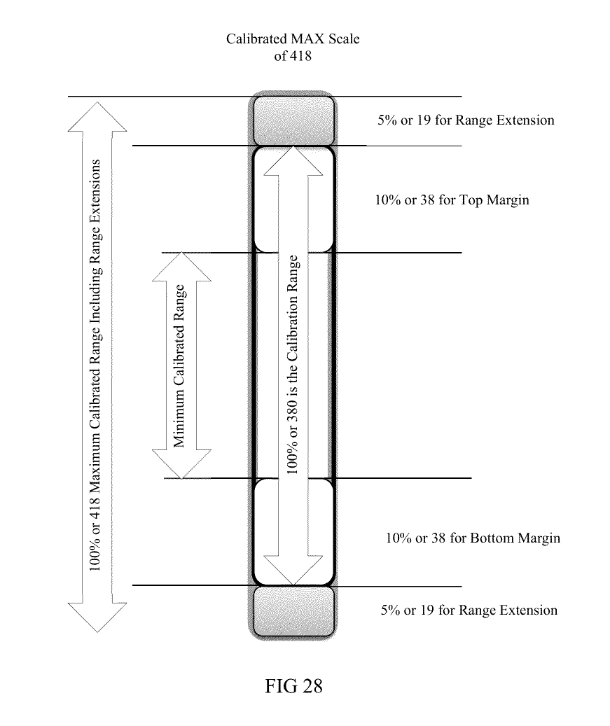

Using the fully-automatic or ‘variable starting point’ calibration method, calibration proceeds similarly to the semi-automatic method except that the user simply performs a normal exercise repetition and the system automatically calculates the repetition's starting position (rather than requiring the user to press a foot switch or start button). A threshold is defined at, e.g., approximately 30% or 40% of the scale (preferably based on a default value for the specific exercise type and machine, and on the user's profile including characteristics such as height and weight), and is preferably also displayed as threshold lines 6 extending (e.g., semi-translucently) across each range-of-motion indicator as shown in FIGS. 12 and 26. When the user selects the exercise to be performed, the threshold lines 6 are displayed at that level, and preferably can be adjusted by the user if desired (e.g., by sliding the adjuster tab 9 up or down). The user then presses a start button on the system to indicate that he/she is ready to perform the exercise/repetition, and gets into position ready to start the power stroke of the exercise. The weight stack can move up or down without the system logging it as the actual start of the exercise as long as the range-of-motion does not exceed the threshold marker location. When the user is ready, he/she simply starts performing the power stroke of the repetition as usual. This causes the range-of-motion indicator to move up the scale, and once it exceeds the threshold, the system continually begins logging the position of the power stroke until the power stroke ends (which can be identified by the range-of-motion decreasing by, e.g., 1%, of the highest value logged so far). The system then begins logging the position of the return stroke until the end of the return stroke (which can be identified by the user beginning the power stroke of the next repetition) and records the lowest position of the return stroke. The system then uses the highest and lowest position values to calculate a calibration factor to re-scale the un-calibrated range-of-motion data for display and storage. For example, if as shown in FIG. 26 the highest recorded position of the calibration stroke was 600 and the lowest was 220, the total un-calibrated range would be 600−220=380. So that the user can still go somewhat past the minimum or maximum of the calibration stroke, an additional range extension such as a fixed value of 5% is preferably added to the top and bottom of the range, extending this un-calibrated range (as shown in FIG. 27) to 418. If the readout covers 0-1024 units, the calibration factor would be 1024/418=2.45. An un-calibrated reading of 418 in this example thus would be displayed all the way up the scale, i.e., 1024, as shown in FIG. 28. Count zones can then be placed on the calibrated range-of-motion indicator, with the lower edge of the lower count zone aligned with the top position of the range extension point, and the upper edge of the upper count zone aligned with the lower edge of the range extension point.

1. A timing window display system for use with a measuring system for measuring force and/or range of an activity, the timing window display system comprising a visual display and configured to visually display on the visual display real-time data from the measuring system during measurement of an activity by the measurement system, the timing window display system further configured to, simultaneously with said visual display of real-time data during measurement of an activity by the measurement system, visually display on the visual display one or more timing windows that move in a predetermined pattern and have a predetermined size.

2. The timing window display system of claim 1, wherein the visual display comprises one or more range indicators.

3. The timing window display system of claim 1, wherein one or both of said predetermined pattern and said predetermined size can be defined at least in part by the type of measured activity, and can be defined at least in part by a user.

4. The timing window display system of claim 1, wherein the measured activity includes a power stroke, a pause, and a return stroke.

5. The timing window display system of claim 4, wherein said predetermined pattern comprises a period of acceleration and a period of deceleration corresponding to said power stroke, and a period of acceleration and a period of deceleration corresponding to said return stroke.

6. The timing window display system of claim 5, wherein the activity comprises a left action and a right action.

7. The timing window display system of claim 2, wherein said timing windows are semi-transparent.

8. The timing window display system of claim 2, wherein said timing windows are colored.

9. The timing window display system of claim 2, wherein said activity comprises an exercise performed by a user in repetitions, and wherein said predetermined pattern of movement comprises positioning each of said one or more timing windows at desired positions with respect to a corresponding range indicator throughout a predetermined period of time.

10. The timing window display system of claim 9, wherein said desired position with respect to a corresponding range indicator comprises said timing window being centered at a leading edge of the desired position of the corresponding range indicator.

11. A system for managing data associated with measured force and/or range of an activity, the system comprising:
a. memory for storing user performance information; and
b. a visual display system configured to visually display real-time data from measurement of an activity and comprising:
i. one or more visually-displayed range indicators; and
ii. one or more visually-displayed timing windows having a predetermined size relative to said range indicators;
the visual display system configured to, simultaneously with said visual display of real-time data, visually display said one or more timing windows in a predetermined pattern of movement associated with said real-time data.

12. The system for managing data of claim 11, wherein said activity comprises an exercise performed by a user in repetitions, and wherein said predetermined pattern of movement comprises positioning each of said one or more timing windows at desired positions with respect to a corresponding range indicator throughout a predetermined period of time.

13. The system for managing data of claim 12, wherein said desired position with respect to a corresponding range indicator comprises said timing window being centered at a leading edge of the desired position of the corresponding range indicator.

14. The system for managing data of claim 13, wherein the measured activity includes a power stroke, a pause, and a return stroke.

15. The system for managing data of claim 14, wherein said predetermined pattern comprises a period of acceleration and a period of deceleration corresponding to said power stroke, and a period of acceleration and a period of deceleration corresponding to said return stroke.

16. The system for managing data of claim 15, wherein the exercise comprises independent left and right sides.

17. The system for managing data of claim 13, wherein one or both of said predetermined pattern and said predetermined size can be defined at least in part by the type of exercise, and can be defined at least in part by a user.

18. The system for managing data of claim 12, wherein the system is configured to store in said memory user performance information that includes the number of times that a range indicator led or lagged the timing window during each repetition.

19. The system for managing data of claim 11, wherein said timing windows are semi-transparent.

20. The system for managing data of claim 11, wherein said timing windows are colored.
